(19) United States
(12) Patent Application Publication
(10) Pub. No.: US 2003/0119014 A1
(43) Pub. Date: Jun. 26, 2003

(54) Biochip

(75) Inventors: Horst Donner, Markt Schwaben (DE); Bernd Drescher, Dossenheim (DE); Andrea Huber, Rosenheim (DE); Jaqueline Weber, Forstinning (DE)

Correspondence Address: RANKIN, HILL, PORTER & CLARK, LLP, 700 HUNTINGTON BUILDING, 925 EUCLID AVENUE, SUITE 700, CLEVELAND, OH, 44115-1405, US

(73) Assignee: MWG-Biotech AG, Ebersberg (DE)

(21) Appl. No.: 10/139,595

(22) Filed: May 6, 2002

(30) Foreign Application Priority Data
May 17, 2001 (EP) 01112179.5

(51) Int. Cl. C12Q 1/68; C12M 1/34

(52) U.S. Cl. 435/6; 435/287.2

(57) ABSTRACT

1. Biochip for the most comprehensive possible detection of the genome of Escherichia Coli K 12.

2.1 The intention is to provide devices to facilitate the most comprehensive possible analysis of the genome of Escherichia Coli K 12.

2.2 A biochip is provided with a multiplicity of probe spots, with a probe applied in each probe spot, each probe having a multiplicity of probe molecules, and all probe molecules of a probe having an identical probe sequence, wherein the probe sequences are nucleotide sequences, and one or more of the probes has probe sequences with a length of at least 20 bases, identical or complementary to a section of an Open Reading Frame of Escherichia Coli K 12.

The invention relates to a biochip.

It is known for biochips to be used to analyse nucleic acids, in order e.g. to understand cellular functions and their regulation. On biochips, sensor molecules are arranged on surfaces in ordered arrays. However, the manner in which these arrays are produced, and the variety of possible applications vary so strongly that it is possible to speak of a multiplicity of different methods. The nucleic acids to be analysed, generally ribonucleic acids (RNA), are isolated from tissue or cells. If RNA is analysed, then enrichment of the mRNA can be effected. The transcription of the RNA and mRNA into cDNA is effected by means of reverse transcriptase. This step usually includes marking of the subsequent hybridisation sample, to make possible detection after hybridisation. With a limited sample quantity, an amplification stage of the original material may be inserted through PCR (polymerase chain reaction) or in-vitro transcription. The data analysis then follows and, in the case of microarray hybridisation, frequently represents the most time-consuming stage, depending on integration density. These biochip technologies have the advantage of integrating the processes of sample preparation, through hybridisation and detection, to data analysis. By running analysis stages in parallel to the greatest possible extent, sample throughput may be increased (Medizinische Genetik [Medical Genetics] No. Mar. 1, 1999 Volume 11, pp 1-32).

An oligonucleotide chip is comprised of a carrier and the molecules needed for the respective experiment, fixed upon it. In the course of production, these molecules are either synthesized on the matrix in-situ by means of photolithographic techniques using physical masks, or else printed on by various methods, such as for example the contact method using capillary needles or the non-contact method based on piezo-electronic ink jet nozzles. The production of printed DNA microarrays divides into the activation and the coating of the solid chip matrix, on to which the biomolecules are fixed by means of suitable coupling chemistry. Basic patent applications relating to DNA chips are e.g. EP 0 619 321 A, EP 0 373 203 A, EP 0 476 014 A and EP 0 386 229 A.

In general, various fluorescent dyes are used as marking agents for nucleic acid analysis. Other means of detection involve the use of radioactive markings, chemiluminescent or other markings, and measurement of the signals in each case detectable with the aid of these markings. Detection is also possible using impedance measurements or other physical methods of measurement. In principle it is also possible to carry out detection based on mass spectrometry using MALDI-TOF analysis (Mass Absorption Laser Desorption Ionization—Time of Flight Spectrometry), in which case marking additives may be dispensed with.

A computer system for the analysis of DNA chips is known from EP 0 923 050 A.

An important basis of biochip technology is hybridisation. Single strands of desoxyribo-(DNA) or ribo- (RNA) nucleic acids are composed of bases such as adenine (A), thymine (T), cytosine (C), guanine (G), uracil (U) or inosine (I). They are able to hybridise into double strands. This involves A linking with T, and C with G, via hydrogen bridges, thus forming so-called base pairs, e.g. AT or CG. There may also be non-complementary base pairs, e.g. AG, GU.

If two different single strands are brought together under suitable conditions containing sufficient adjacent complementary base pairs, they hybridise into a double strand nucleic acid. Under suitable conditions, DNA/DNA, DNA/RNA and RNA/RNA hybrids are obtained in this way.

The hybridisation of nucleic acids finds application in the detection of certain nucleic acid sequences of sample material, which must be prepared in advance. This involves the provision of nucleotide sequences complementary to the sought nucleic acid sequence, and which hybridise with the sought sequence. The formation of such a double strand must then be detected by suitable methods. Such a nucleotide sequence, which is applied to the surface of a carrier, may consist of DNA or cDNA sections selectively amplified by PCR techniques, which are specific for a certain transcript of an organism.

A cDNA chip or a biochip with nucleic acid sequences, in which probe material obtained by PCR techniques is applied to the carrier, has the following disadvantages:

the sequence is generally selective only over a small part of its length,

because of the PCR techniques it frequently occurs that the sequences contain unforeseen nucleotides in places,

the physical parameters of the various sequences on such a biochip, such as e.g. the melting temperature, may be standardised only within a narrow framework, since the lengths of the respective amplification products are not always freely selectable,

owing to incorrectly incorporated nucleotides and physical probe properties which have not been optimally standardised, varying amounts of sample material are used in order to obtain detectable signals,

because of the aforementioned sources of error, signals obtained are hard to compare with one another, since the errors may be of varying extent from one probe to another,

experiments in which it is intended to use such biochips for the production of gene expression patterns, lead to results which are not very exact because of the drawbacks listed above.

Biochips on which (synthetic) oligonucleotides are applied, on the other hand, have the following advantages:

the length of sequences is freely variable,

the sequence may therefore be selective over a maximum length,

such oligonucleotide sequences are generally shorter than e.g. sequences stemming from biological material amplified by means of PCR.

Since a probe has many identical probe molecules, and short sequences are synonymous with short probe molecules, probes with oligonucleotide sequences react more reliably with sample material than longer probe molecules, which tend rather to hybridise with themselves or the adjacent molecule, or to align themselves unevenly on account of their length. Hybridisation experiments with oligonucleotides are therefore more reliable, more uniform and less complicated.

Oligonucleotide chips exist with in-situ synthesised oligonucleotides. Such a method is very laborious. It involves building up the oligonucleotides step by step, with one nucleotide after the other being added to the developing oligonucleotide by means of masks. The longer the molecule, the more flexible it is, and the more unreliable in contrast the masks appear to function, so that biochips produced by this method can be made reliably only with oligonucleotides with comparatively few bases.

Longer oligonucleotides, however, may also be synthesised ex-situ, spotted on to a carrier and fixed to it covalently or by other means. Biochips produced in this way do not have the drawbacks described above and are obtainable commercially for individual applications. A feature of ex-situ or ex vivo synthesis is the scope for quality control, e.g. by means of MALDI-TOF analysis (Mass Absorption Laser Desorption Ionization—Time of Flight Spectrometry). By this means, in contrast to direct synthesis on the carrier (e.g. by photolithographic methods), oligonucleotides of high purity may be guaranteed.

In hybridisation experiments, either the nucleic acid sample to be analysed or the oligonucleotide or nucleic acid sequence complementary to the sought nucleic acid sequence is immobilised in a solid phase. After successful hybridisation the product can be washed. Identification is generally made using radioisotopes, dyes or other marking agents incorporated during or after hybridisation, and which ultimately allow the subsequent detection of a signal by physical means (C. R. Cantor, C. L. Smith, Genomics, John Wiley and Sons, New York 1999, p. 67). In this way many individual analyses may be made in parallel on so-called chips, thereby increasing the possible sample throughput per unit of time.

The analysis of biological sample material by reaction with biochips leads to molecules immobilised on the chip surface. Their topology and quantities on the biochip allow statements on the amount of a particular nucleic acid sequence, a gene or a gene product in this sample material. From this it is possible to derive biological information e.g. concerning gene expressions and mutations in a genome. These have a potential accelerating benefit in the development of new medicines, in diagnosis and other areas of biomedical and biochemical research (M. Schena, R. W. Davis, Genes, genomes and chips in DNA Microarrays, published M. Schena, Oxford University Press 1999).

In order to detect a hybridisation without error, it must be sufficiently stable and specific under the usual relevant conditions.

The stability of a nucleic acid double strand is expressed in its melting temperature. The melting temperature is the temperature at which a potential hybrid is present in equal parts in double strand and single strand form. It depends on the nature and composition of the base pairs from which a double strand is composed, together with the nature and composition of the medium in which the double strand is present.

The specificity of a hybridisation, i.e. the percentage of hybrids which have no defective bond in the sense of non-complementary base pairs, is closely dependent on the stability of the hybrids formed. Adjacent complementary base pairs, depending on their respective nature and composition, contribute to the overall stability of a hybrid. If, within a hybrid, there is an imperfect bond in the form of a non-complementary base pair then, in comparison with a hybrid formed with no defects, the contribution of a base pair together with two cooperative interactions of two adjacent bonded base pairs to the enthalpy of formation of that incorrectly-formed hybrid are lacking. This is expressed in the lower melting temperature of the malformed hybrid as compared with hybrids with no imperfect bonds. Thus for example a fully complementary DNA/DNA-14 mer has a melting temperature roughly 7° C. higher than a 14 mer with an imperfect bond (C. R. Cantor, C. L. Smith, Genomics, John Wiley and Sons, New York 1999, chap. 3). A content of 1% of imperfect bond in a given hybrid lowers its melting temperature by around 1° C. Consequently the temperature chosen for carrying out a hybridisation is, together with the nature and composition of the reaction medium, a control parameter for the specificity of a hybridisation (R. J. Britten, E. H. Davidson, Hybridisation strategy in nucleic acid hybridisation: a practical approach, pub'd. B. D. James, S. J. Higgins, IRL Press 1985).

The specificity of a hybridisation also depends on the length of the base fragments entering into the hybrid. The more adjacent base pairs hybridise, the faster the actual hybridisation proceeds. An imperfect bond, on the other hand, slows down the formation of a hybrid. This difference in speed increases with the number of base pairs forming a hybrid. 10% of imperfect bonds in a given hybrid generally slow down the reaction rate by half (R. J. Britten, E. H. Davidson, Hybridisation strategy in nucleic acid hybridisation: a practical approach, pub'd. B. D. James, S. J. Higgins, IRL Press 1985).

If DNA single strands are able to form eight or more base pairs then, because of the achievable specificity and the bonding strength of the individual base pairs, it is certain that perfect double strands will be formed in the overwhelming majority of hybridisations (C. R. Cantor, C. L. Smith, Genomics, John Wiley and Sons, New York 1999, p. 11).

Examples of applications for hybridisation experiments are the detection of Mycoplasma Pneumoniae (EP 0 173 920 A2), the detection of the protein human telomerase reverse transcriptase (hTRT) (EP 0 841 396 A1) and the detection of certain polymorphisms (e.g. EP 0 812 922 A2).

Another important area of application for biochips is the analysis of gene expression patterns. Gene expression patterns allow a statement on the expression of specific genes of an organism depending on parameters which are varied according to the experiment. For this purpose it is in principle desirable to be able to analyse on a biochip the degree of expression of as many different genes as possible at the same time. In this way it is possible to minimise the time needed to analyse the gene expression of as many genes of a genome as possible.

Qualitative statements on the degree of expression of different genes of a genome may thus be made with a high degree of certainty, since statements on the degrees of expression of many different genes of a genome can be made with the aid of an analysis experiment under conditions which are to the greatest possible extent identical. In contrast to this, statements on the expression of the same genes of a genome made with the aid of analysis experiments which might be subject to varying conditions, would be less meaningful. This would be the case if one were to analyse the same number of gene expressions on different biochips, or if the same number of gene expressions were to be determined with probes or measuring methods of different kinds.

In order to obtain meaningful gene expression patterns of as many different genes as possible using a single biochip, it is necessary to ensure that the oligonucleotide sequences applied to the surface of the biochip are suitable for specific hybridisation with an expression product which can be assigned to a specific gene.

As explained above, the specificity of hybridisations depends, in addition to the complementarity of the base sequences which form the hybrid, on the length of the hybrid, its individual composition and its melting temperature.

If the expression of many genes of a genome is analysed at the same time, then consequently it is necessary for many sequences to have similar properties such as length and melting temperature, for the results to be compared with one another.

If the expression of a few genes of a genome is analysed, then correspondingly fewer sequences require similar properties such as length and melting temperature.

Nevertheless it is important that a probe sequence on a biochip specifically detects the expression of a gene from the overall total of expressed genes of an organism.

In order to make such probe sequences available it is necessary for the genome of the organism to be analysed to be largely known.

In the genome of an organism, so-called Open Reading Frames (ORFs) may be identified. An ORF is an area within a DNA molecule, from which it is assumed on the basis of a specific start sequence and a specific end sequence that it is in principle capable of being expressed by natural means.

An organism with a known genome is Escherichia Coli, a bacterium of the Enterobacteriaceae family. The presence of Escherichia Coli and other bacteria in the gut flora is necessary for the physiological functions of the digestive system. Besides the non-pathogenic Escherichia Coli strains there are also in existence Escherichia Coli strains which are infectious to human beings. Escherichia Coli has also served for a long time as a model organism in the study of fundamental cellular and molecular processes, and for the study of reactions by which bacteria react to environmental factors, physiological changes or differentiation factors. Populations of Escherichia Coli are also of great importance for the clinical production of amino acids and complex enzymes and proteins such as insulin, in addition to being an important source for the production of K and B complex vitamins.

Among the Escherichia Coli strains, Escherichia Coli strain K 12 is established as a widely distributed model organism, used as reference strain for gram negative bacteria. In order to make it possible to study the behaviour of the model organism under various conditions (nutrient, temperature, etc.) and/or to compare other organisms with it, it should be possible to analyse the expression of as many genes of an organism as possible simultaneously.

Richmond et al. (Nucleic Acids Research, vol. 27, No. 19, 1999, 3821-3835), Arfin et al. (Journal of Biological chemistry, vol. 275, No. 38, 29672-29684) and Dong et al. (Applied and Environmental Microbiology, vol. 67, No. 4, 1911-1921) disclose for this purpose DNA arrays in which PCR products representing ORFs of E. Coli K 12 are spot-deposited on a carrier. The use of PCR products for such a chip has the drawbacks described above (low selectivity, low specificity, time-consuming to create, etc.). Because of their length, such probes often tend to give faulty hybridisation.

Ten Bosch et al. (Data Base Biosis online Biosciences Information Service, Philadelphia Pa., US; International Genome Sequencing and Analysis C, “validation of sequence optimised 70 base pair oligonucleotides for use on DNA microarrays”) describe the selection of oligonucleotides which are specific for ORFs of Saccaromyces Cerevisiae, but without describing in this paper the selection method used.

Lipshutz et al. report in Nature Genetics supplement, vol. 21, 1999, 20-24 on in-situ oligonucleotide chips produced by photolithographic methods, and announce a corresponding E. Coli K 12 chip. In-situ oligonucleotides are afflicted with the disadvantages described above (production which is time-consuming and prone to error, limited length of probe molecules). In addition

due to the masks used in the photolithographic method of production, one is restricted to specific layouts of probes on the chip,

by-products from chain-terminating reactions remain stuck to the chip and may falsify the results,

the comparatively short probes are not very sensitive, since only complements can be detected, but their presence in adequate quantities is not always guaranteed (e.g. poor in the event of incomplete amplification of the sample material), and

due to the low number of possible permutations, short probes have a relatively low specificity.

The problem of the invention is therefore to provide a device for the most comprehensive possible detection of the genome of Escherichia Coli K 12, which overcomes the drawbacks of known devices.

The problem is solved by a biochip according to claim 1, in which probes suitable for the specific detection of genes and the activity of genes of Escherichia Coli K 12 respectively are provided on a carrier.

In the biochip according to the invention the probe sequences are nucleotide sequences with a length of 30-80 bases, made from ex-situ synthesised oligonucleotides, wherein one or more of the probes has probe sequences which are identical or complementary to a section of an Open Reading Frame of Escherichia Coli K 12.

The presence of ex-situ synthesised oligonucleotides as probe molecules has the following advantages, amongst others, over PCR products:

the homogeneity of probe molecules in a probe spot can more easily be ensured, since oligonucleotides are easy to purify and their synthesis is relatively uncomplicated, since e.g. no specific primers are needed,

as compared with PCR products, which are frequently over 100 bases long, comparatively short probe molecules are available. These have enhanced specificity, since long probe molecules often lead to faulty hybridisation on account of the higher number of base sequences they contain, which are able to enter a hybridisation.

In comparison with a biochip with in-situ synthesised oligonucleotides, a chip according to the invention has the following advantages, amongst others:

it is easier to ensure probe purity since, with in-situ synthesis, faulty reactions such as chain terminating reactions remain as faults on the chip, whereas such by-products of ex-situ synthesised oligonucleotides may removed before spot-deposition,

the quality control of ex-situ synthesised oligonucleotides before spotting-on simplifies the production of such chips,

with a length of at least 30 bases, probe molecules according to the invention are longer than many oligonucleotide probes provided on in-situ chips, and are therefore more sensitive than such probes, since there is a longer base sequence available which can hybridise to the sample material, and

the oligonucleotides provided on the chip may be more specific than in-situ oligonucleotides of lesser length, since the number of permutable bases in the respective probes is higher than in conventional in-situ chips, which typically involve 25 mer.

A biochip thus formed also has the advantages that the length of the probes may be precisely varied from specific standpoints, that it is especially easy to ensure high purity and thus selectivity and reactivity of the probes, together with reproducibility of experiments conducted with a single biochip, and that it has probe molecules which are specific for Escherichia Coli K 12.

According to a preferred embodiment, probes of a biochip according to the invention have an area which is respectively identical or complementary to a section of the sequence sections SB 1- SB 12812.

According to another preferred embodiment, probe sequences of a biochip according to the invention are identical or complementary to a section of the core zone KB 1-KB 12812.

According to a preferred embodiment, the probe sequences of a chip according to the invention have base lengths between 40 and 60 bases.

According to a preferred embodiment the biochip has 4289 specific probes for 4289 different genes of the bacterium Escherichia Coli K 12. Such an embodiment has the advantage that a complete set of genes of the genome of the model organism Escherichia Coli K 12 is provided, and with this a single device is used to analyse the expression of completely different genes simultaneously.

A further preferred embodiment of a biochip according to the invention is a biochip which, apart from probes for genes of the bacterium Escherichia Coli K 12, also has probes for mutants of Escherichia Coli K 12 and/or for other Escherichia Coli strains.

Such biochips have the advantage of making possible a comparison of gene expression in the model organism Escherichia Coli K 12 and in the corresponding mutants and/or other Escherichia Coli strains, in one and the same hybridisation experiment. In addition it is possible with such a biochip to make not only qualitative and quantitative statements regarding the respective gene expression but also statements concerning the population density of the measured Escherichia Coli strains or mutants. Such a biochip may also be used for diagnostic procedures, e.g. when it is a question of determining which Escherichia Coli strain is present in the gut flora of a patient.

Another preferred embodiment of a biochip according to the invention is a biochip which, besides probes for genes of the bacterium Escherichia Coli K 12, also contains probes for other bacteria than Escherichia Coli K 12. Such a biochip has the advantage of being usable for diagnostic purposes since, in addition to the presence of the Escherichia Coli K 12 strain in a sample, the presence of other bacteria can also be detected.

Other advantageous variants are disclosed in the subsidiary claims.

A selection procedure ensures that the probe sequences hybridise specifically with probe material which can be assigned to a specific gene or ORF of the Escherichia Coli K 12 type.

Depending on the expression products which are to be used to produce the expression sample, the probe sequences are identical or complementary to sequence sections from the genome of Escherichia Coli K 12.

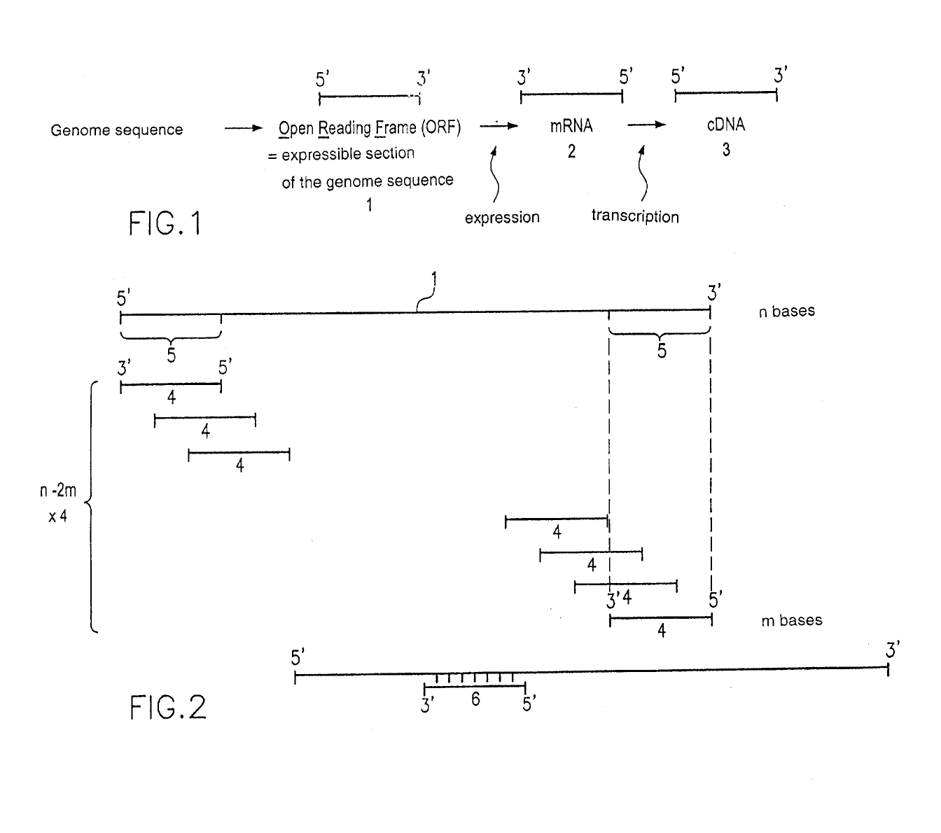

The invention is described below with the aid of FIGS. 1-5, showing in

FIG. 1 the generation of cDNA,

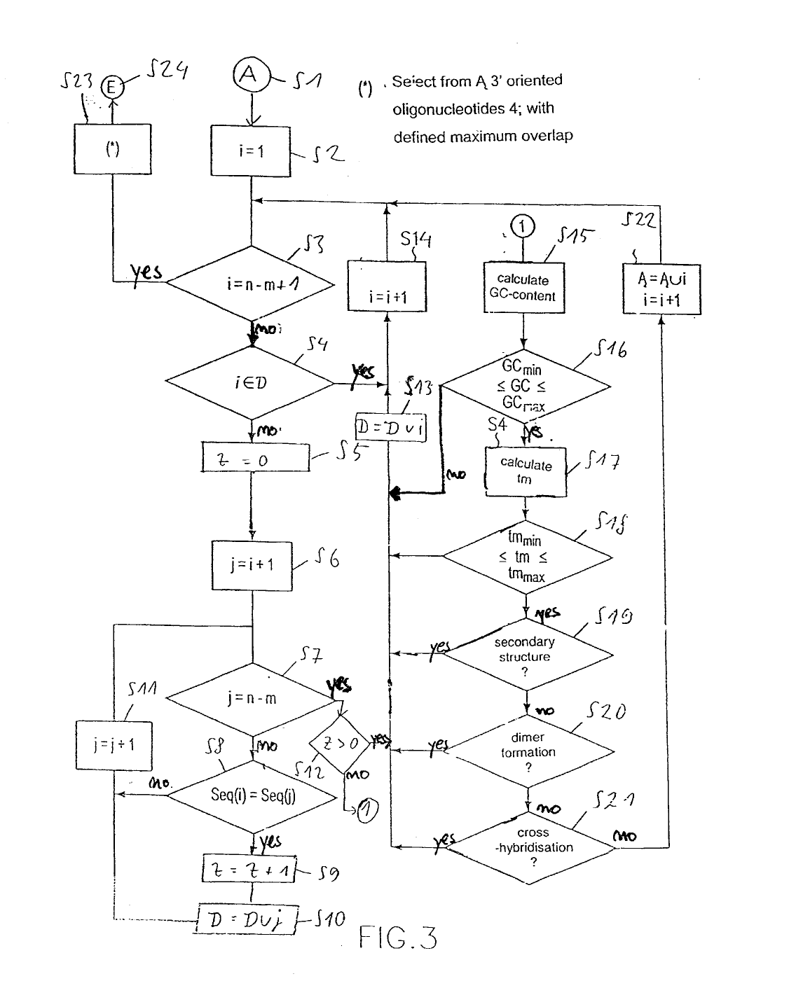

FIG. 2 an ORF and a cDNA molecule respectively, and complementary probe sequences,

FIG. 3 the selection procedure according to the invention in a flow diagram,

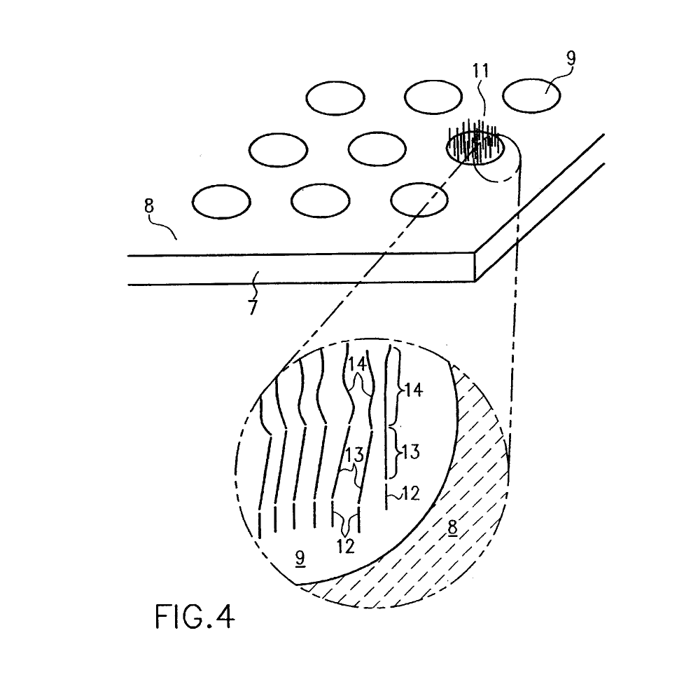

FIG. 4 a schematic section of the biochip according to the invention, and

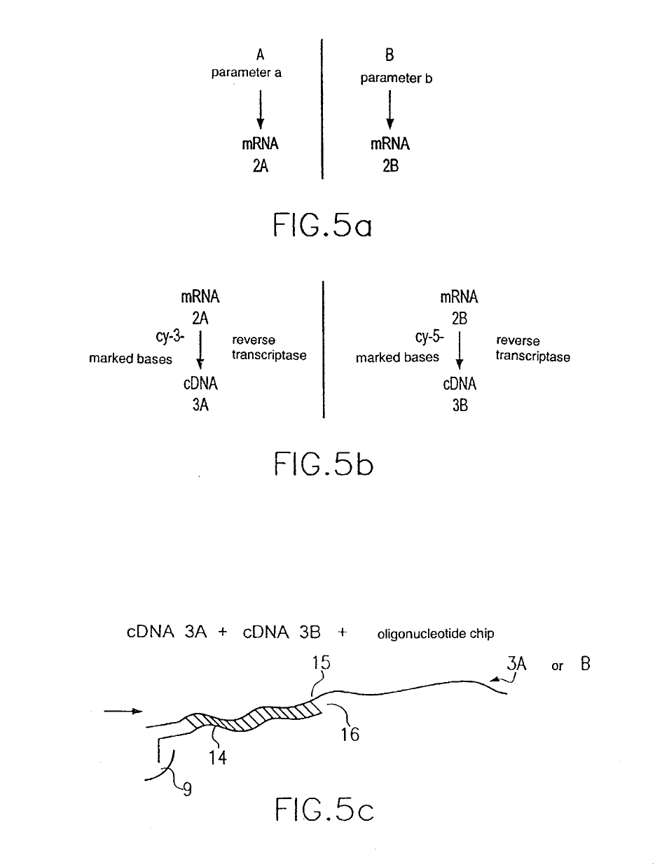

FIGS. 5a)-c) in each case schematically the sequence of the reaction steps executed on the biochip according to the invention

The biochip according to the invention facilitates the analysis of gene expressions of Escherichia Coli K 12. This involves identifying which genes of Escherichia Coli K 12 have been expressed and to what relative extent.

A sequence section from the genome of an organism which is expressed represents a gene. This generally involves an ORF 1 (Open Reading Frame). If an ORF 1 is expressed in one step, then from this gene or ORF 1 a certain amount of mRNA 2 is produced, with this amount being dependent on various parameters (FIG. 1).

In the production of a gene expression sample it is established whether or not mRNA 2 has been produced and if so, to what extent this has o mRNA 2 produced is processed by reverse transcription of the mRNA 2 into cDNA 3, with e.g. a fluorescent cy-3 or cy-5 marker being

With a cDNA 3 marked in this way it is possible, in combination with a biochip according to the invention, to make a statement on the amount of expressed mRNA 2. This is explained below.

For this purpose the cDNA 3 is combined with a biochip according to the invention on which are applied nucleotide sequences which are able to hybridise with the cDNA 3 molecules.

Such specific sequences may be identified, amplified from biological material by means of PCR, and applied to a biochip.

A preferred embodiment of the invention is one in which synthetic oligonucleotides are used to produce the probes of a biochip.

The oligonucleotide sequences used here are selected with the aid of computerised calculations.

1. Computerised Selection Process for the Selection of Probes

This selection process is explained below with the aid of FIGS. 2 and 3.

Firstly a specific length of m bases is prescribed for the oligonucleotide sequences which should form the major portion of probes of an oligonucleotide chip according to the invention.

An ORF 1 which is n bases long can contain a sequence section 5 which is m bases long. An ORF 1 which is n bases long has n−m+1 different sequence sections 5 with a length of m bases. Accordingly, n−m+1 different oligonucleotide sequences 4 may be identified; these are complementary to n−m+1 sequence sections 5 of an ORF 1 and have a length of m bases.

For each ORF 1 of the Escherichia Coli K 12 genome with a length of n bases, wherein n may vary for each ORF 1 but n is greater than m, the n−m+1 oligonucleotide sequences 4 which are complementary to n−m+1 possible sequence sections 5 of an ORF 1 of Escherichia Coli K 12 are identified (FIG. 2).

At the same time quantities D, A are defined and the empty quantities Ø (D=Ø and A=Ø) are assigned to them. The quantity D contains all deleted oligonucleotide sequences, and the quantity A contains all tested and accepted oligonucleotide sequences.

The process begins with step S1 (FIG. 3). In the subsequent step S2 a counter i equal to 1 is set. The counter i stands for the i-th oligonucleotide sequence which is to be tested to establish whether it is specific for the ORF, i.e. testing for an i-th oligonucleotide sequence 4i (i=1, 2, 3, . . . n−m+1) and i∉D, whether oligonucleotide sequences 4ⱼ exist with sequence (4ᵢ)=sequence (4ⱼ)i, wherein j≠i and j=(i+1, 2 . . . , n−m+1).

The process sequence then moves to query S3 which is used to check if the counter i is equal to n−m+1.

If the query in step S3 reveals that i is not equal to n−m+1, then the process sequence moves on to query S4, which checks whether i is an element of the quantity D. If this is not the case, then a further counter z equals 0 is set (step S5).

In the subsequent step S6 a further counter j equals i+1 is set. The counter j describes the j-th oligonucleotide sequence.

Step S7 involves checking as to whether j is identical to n−m+1. If the answer is no, then in step S8 a check is made as to whether the oligonucleotide sequence 4ᵢ is identical to the oligonucleotide sequence 4ⱼ. If this is the case, then in step S9 the counter z is increased by the value 1 and in step S10 the quantity D is added to j, meaning that the oligonucleotide sequence 4ⱼ is deleted. The process sequence then moves on to step S11, in which j is increased by the value 1. If the query in step S9 reveals that the two sequences are not identical, then the process sequence moves directly to step S11. From step S11 the process sequence returns directly to step S7.

The process steps S7 to S11 form a loop, which is passed through for all j equal i+1 to j equal n−m+1. If the counter j equals n−m+1, then in step S7 the process sequence branches to step S12 which involves checking whether the counter z is greater than 0.

If this is the case, then the process sequence branches to step S13 in which the quantity D is added to the value of i. Then in step S14 i is increased by the value 1 and the process sequence returns to step S3, after which steps S4, S5, etc. are repeated. If the query in step S4 reveals that i is already an element of D, then the process sequence moves directly to step S13 in which i is increased by the value 1.

If the query in step S12 reveals that z is equal to 0, i.e. that no j-th oligonucleotide sequence 4ⱼ with j=(i+1, 2 . . . n−m+1) exists which is equal to the i-th oligonucleotide sequence 4ᵢ, then the process continues with step S15.

In step S15 the guanine and cystosine content (GC content) is determined for an i-th oligonucleotide sequence 4ᵢ (i=1, 2, 3, . . . n−m+1) and i ∉ D, relative to all bases of this sequence.

In the subsequent step S16 the oligonucleotide sequence 4ᵢ is tested to establish whether the GC content (GC) lies within a predetermined range (between GCₘᵢₙ and GCₘₐₓ). This range may for example be between 40 and 60%.

If the result is negative, skip to step S13 in which the oligonucleotide 4ⱼ is deleted.

If the result is positive, continue with step S17.

Step S17 involves calculation of a melting temperature tm for an i-th oligonucleotide sequence 4ᵢ (i=1, 2, 3, . . . n−m+1) and i ∉ D.

In step S18 the oligonucleotide 4ᵢ is tested to establish whether the calculated melting temperature tm lies within a predetermined range. This range may for example be between tmₘᵢₙ=65° C. and tmₘₐₓ=75° C.

If the result is negative, skip to step S13 in which the oligonucleotide 4ᵢ is deleted.

If on the other hand the result is positive, then step S19 is taken. This involves testing whether the oligonucleotide 4ᵢ can form secondary structures which make hybridisation of the oligonucleotide 4ᵢ with a complementary sequence section 5ᵢ difficult or improbable. If this is the case, branch to step S13 in which the oligonucleotide 4ᵢ is deleted.

If on the other hand the answer is negative, then continue with step S20 in which it is checked whether the oligonucleotide 4ᵢ can form dimers. Which make hybridisation of the oligonucleotide 4ᵢ with a complementary sequence section 5ⱼ difficult or improbable. If this is the case, branch to step S12 in which the oligonucleotide 4ᵢ is deleted.

If the answer on the other hand is positive, then in step S21 a test is made as to whether cross-hybridisation is possible between the oligonucleotide 4ᵢ and a sequence section 5ⱼ from a different ORF 1 than that containing the sequence section 5ⱼ used originally to identify the oligonucleotide 4ᵢ. If the answer is yes, branch to step S13.

If the answer is negative, continue with step S22 in which first of all i is added to the quantity A, meaning that the oligonucleotide 4ᵢ is accepted (A=A ∪{4ⱼ}), after which the counter i is increased by the value 1.

The process sequence then reverts to step S3. If the query in step S3 reveals that i is equal to n−m+1, then the process sequence branches to step S23 in which, from the quantity of oligonucleotides 4ᵢ ∈A for example 3 oligonucleotides are so selected that they hybridise closest at the 3′-end of the ORF 1 and overlap one another by a preset percentage to the maximum extent.

This selection process is terminated with step S24.

The above selection process is undertaken for all identified ORFs of Escherichia Coli K 12.

Within the scope of the invention it may also be expedient to select sequence sections 5ⱼ instead of oligonucleotide sequences 4ᵢ. This may be the case if it is desired to detect, using an oligonucleotide chip according to the invention, mRNA 2 instead of cDNA 3. Steps S1 to S12 may therefore be taken analogously for the sections 5ᵢ.

Steps S16, S17, S18, S19, S20 and S21 represent advantageous criteria for the selection of oligonucleotide sequences which, however, may also be omitted or used in different combinations.

This method can also be applied to other ORFs than ORFs from within the Genome of Escherichia Coli K 12.

2. Structure of a Biochip According to the Invention

A cut-out section of a biochip according to the invention is shown in FIG. 4. A biochip according to the invention is a carrier 7 with a carrier surface 8 on which probe spots 9 are applied in lines and rows in an ordered array, in which each probe spot 9 has a multiplicity of identical probe molecules 1 0. The probe molecules 10 of a probe spot 9 form a probe 11. Each probe molecule 10 is comprised of a linker 12 which facilitates a covalent bond of the probe molecule to the carrier surface 8, a spacer 13, and a probe sequence 14.

A 5′-amino-modified oligonucleotide at the 5′-end of the spacer 13 is a linker 12.

The spacer 13 allows the maintenance of a distance between the carrier surface 8 and the probe sequence 14. A suitable spacer is for example a C₆-oligonucleotide made up of six dCTP monomers.

5′-amino-modified oligonucleotides are provided which have between the amino-modification representing the linker 12 and the probe sequence 14 a C₆ spacer 13 which is comprised of six dCTP monomers. In production of the biochips, the oligonucleotides are dissolved to a concentration of 100 μM in 50 μM Na-borate (pH 8.5) and 250 mM NaCl and spot-deposited on silylated slides of type CSS of CEL Associates, Houston USA, using an Affymetrix 417 arrayer. They are then rehydrated in a moisture chamber and dried at room temperature. Finally the formation of the covalent bond between linker and carrier surface is concluded by a reduction with NaBH₄.

An oligonucleotide sequence 4 selected with the aid of the process described above may be a probe sequence. The probe sequence is bonded by the 5′-end to the spacer 13. The probe sequences 14 of a probe 11 differ from the probe sequences 14 of other probes 11. Only identical probes 11 are provided within a probe spot 9. Within the scope of the invention it is however also possible to provide different probe sequences 14 within a probe 11.

Within the scope of the invention it is also possible to provide different spacers, linkers, carrier surfaces and types of covalent bond, just as it is possible to arrange probes on a carrier by means other than spot-deposition.

3. Typical Embodiment

By the means described under 1. above, an oligonucleotide sequence 4, applied as probe sequence 14, is determined for each of 4289 ORFs in the genome of Escherichia Coli K 12.

The determined probe sequences 14 are complementary to sequence sections 5 from the genome of Escherichia Coli K 12, as given in the appended sequence listing.

The sequence listing is available both in paper form and in the form of an electronic file stored on a machine-readable data carrier. The numeric identifiers in pointed brackets<> used in the protocol correspond to the numeric identifiers of WIPO standard no. 25, with definitions as follows;

<160> the total number of sequence zones within which lie determined sequence sections 5, the complement of which may be applied as probe sequence 14 to an oligonucleotide chip according to the invention,

<210> in each case a sequence zone SB within which a sequence section 5 may lie; assigning a sequence identifier to each sequence zone,

<220> the section of the genome of Escherichia Coli K 12 to which the sequence zone

<210> is to be assigned; this section at the same time corresponds to one of 4289 ORFs from the genome of Escherichia Coli K 12,

<222> the position of the sequence zone <210> within the section <220>,

<223> the position of the section <220> within the genome of Escherichia Coli K 12, and

<313> a core zone of the respective sequence zone <210>, the complement of which is a constituent of a probe sequence 14 (the position of the core zone <313> within the respective section <220>);

In the sequence listing, in each case up to three sequence zones <210> are specified for 4289 different ORFs 1, with the up to three sequence zones <210> specified for one ORF 1 (<220>) in each case differing from all other specified sequence zones <210> of the other ORFs 1 (<220>) of the genome of Escherichia Coli K 12. The sequence zones <210> belonging to one ORF 1 are shown directly after one another in the sequence listing.

A section of a predetermined length of one of the sequence zones <210> is specific for the ORF 1 concerned. In the present example the minimum length is 30 bases.

Preferred as probe sequences are sections which comprise the 20 bases of the core zones as specified under numeric identifier <313> of the respective sequence zones <210>. These core zones or their complements form especially preferred oligonucleotide sequences 4 and probe sequences 14 respectively (FIG. 4), since they are specific.

In principle it is also possible to use as probe sequences in particular even longer zones comprising only a part of the core zones <313> or no part of the core zones <313>. As a rule such sequences are also specific if the number of bases is at least 40.

For a simplified presentation, the sequence zones <210> will be shown below by SB (Sequenzbereich [=sequence zone]) followed by their sequence identifier shown in the sequence listing after the numeric identifier <210>, and the core zones <313> by KB (Kernbereich [=core zone] followed by the sequence identifier of the associated sequence zone.

In the embodiment according to the invention, with the aid of the sequence listing, 4289 oligonucleotide sequences 4, which are 40 bases long, are selected. These have passed through the checking process shown in FIG. 3 and are complementary to 4289 sequence sections 5 of the first sequence zones of the respective ORFs 1 (<220>) specified in the sequence listing, wherein each oligonucleotide sequence 4 is complementary to a sequence section 5 of the respective ORFs 1 (<220>) which commences 9 bases before the respective core zone KB and ends 11 bases after this sub-section.

A total of 4416 probes are applied to a carrier. 4289 of these probes have probe sequences 14 which are 40 bases long and conform to the 4289 oligonucleotide sequences 4 described above. The remaining 127 probes are control probes. 79 of these are replicates of complements of sections of the genome of Escherichia Coli K 12 which always hybridise (positive control). The other 48 probes are probes with probe sequences which are unsuitable for hybridisation with a transcription product from the expressed genome of Escherichia Coli K 12 (Arabidopsis negative controls).

Each of the 4289 probes hybridises with a different cDNA molecule 3 which is reverse transcribed from the mRNA 2 respectively expressed from an ORF 1 (<220>).

The probes are applied to the carrier on a surface of 2×2 cm² as described above.

Within the scope of the invention, probe sequences 14 other than those of oligonucleotide sequences 4 specified in the embodiment may be applied to the biochip, so long as they are part of a sequence zone <210> specified for the respective ORFs 1 (<220>).

An advantageous embodiment of a biochip according to the invention is a biochip on which are provided probe sequences for the detection of sample material (e.g. cDNA 3 or mRNA 2) of Escherichia Coli K 12 cultures, and on which additional probes are provided for the detection of mutations of Escherichia Coli K 12 within this sample material.

An advantageous embodiment of a biochip according to the invention is a biochip on which are provided probe sequences for the detection of sample material (e.g. cDNA 3 or mRNA 2) of Escherichia Coli K 12 cultures, and on which additional probes are provided for the detection of other bacteria strains within the sample material.

This opens up more extensive selection options for special versions of a biochip according to the invention. If it is desired for example to analyse the expression of a specific gene group on a preferred basis, then the relevant probe sequence may be derived as described above from the sequence listing, from the sequence zones for this gene group, and optimised separately in the experiment or in a calculation.

For example the free selection of the length of the probe sequences 14, the preferred melting temperature of the calculated hybrids 6, and the calculation programs used for the calculations concerned permits more accurate matching.

Accordingly it may also be expedient to provide oligonucleotide sequences of different lengths in individual probe spots.

Within the scope of the invention, however, other types of assignments of gene groups may also be made.

A further possible means of optimisation lies in an experimental comparison of different probe sequences 14 selected for an ORF 1 by the method described above.

4. Example of the Use of a Biochip According to the Invention

The use of a biochip according to the invention in a method for the analysis of a gene expression pattern of Escherichia Coli K 12 is described below with the aid of FIGS. 5 a)-c).

Two nutrient solutions A and B are made with Escherichia Coli K 12, with nutrient solution A being subject to different conditions from those of nutrient solution B. The different conditions involve the parameters, the effect of which on the gene expression is being analysed. Such parameters may for example be temperature, concentration and/or special contents of the nutrient solutions (FIG. 5a)).

In a first step the respective mRNA 2 of nutrient solutions A and B is transcribed into cDNA 3, with cy-5 marked bases being incorporated in the one cDNA transcript and cy-3 marked bases being incorporated in the other cDNA transcript FIG. 5b)).

The two cDNA samples 3A and 3B are brought into contact with a biochip according to the invention (FIG. 5c)).

Those cDNA molecules 3 which have a sequence section 15 which is complementary to a probe sequence 14 which is present on the biochip, form a hybrid 16 (FIG. 5c)).

Those cDNA molecules 3 which have no sequence section 15 which is complementary to a probe sequence 14 which is present on the biochip, or which are not in spatial proximity on the biochip to a probe sequence 14 which is complementary to such a sequence section 15, are removed by washing from the surface of the biochip.

The amount of hybrid 16 formed in each individual probe spot 8 may now be detected with the aid of a fluorescence spectrometer. This involves measuring the intensity of the fluorescence. This occurs at a wavelength which is specific for the marker concerned. If an ORF 1 of Escherichia Coli K 12 is expressed only in one of the two nutrient solutions A or B, then fluorescence is detected only at the wavelength which is specific for the marker incorporated during synthesis of the corresponding cDNA 3. If an ORF 1 of Escherichia Coli K 12 is expressed in both nutrient media, then fluorescence will be detected at wavelengths specific for the marker used in the synthesis of cDNA 3. The markers used in the embodiment are cy-3 and cy-5. These two markers fluoresce at different wavelengths. It is therefore possible, in each individual probe spot with a detectable minimum quantity of hybrid 16, to determine an intensity value for the fluorescence broken down by wavelength. Measurement of the fluorescence is therefore effected in two so-called channels:

a cy-3 channel, used to measure the fluorescence intensity of cy-3 marked cDNA 3A which has formed hybrids 16A on the biochip with probe sequences 14, and

a cy-5 channel, used to measure the fluorescence intensity of cy-5 marked cDNA 3B which has formed hybrids 16B on the biochip with probe sequences 14.

The fluorescence intensity of each individual probe spot 8 is measured in the relevant channel. Also measured is the so-called background noise, i.e. the fluorescence intensity of the biochip surface in zones between the probe spots 8. The difference between these two measured values gives a corrected value for the fluorescence intensity of the cDNA molecule 3 which has formed a hybrid 16 in the measured probe spot and which is provided with the relevant marker. This corrected intensity value is proportional to the quantity of ORF 1 expressed from Escherichia Coli K 12 in one of the two nutrient media, for which the measured probe 11 is specific.

In the measuring channel in which are determined the values which lead to this corrected intensity value, it is specifically the intensity of fluorescence of a marker be corresponding to the measuring channel which is measured, and at the same time its relative concentration. If the corrected intensity value is above 0, or if the intensity of the fluorescence measured in a probe spot lifts sufficiently from the background, then there is qualitative proof of the existence of cDNA 3 provided with a specific marker in the relevant probe spot 8.

The totality of intensity values determined in a channel corresponds to a gene expression pattern, which gives information on the expressions of those ORFs 1 of Escherichia Coli K 12 for which specific probes 9 are provided on the biochip. The gene expression pattern is dependent on the conditions to which the relevant sample of Escherichia Coli K 12 was subject before the transcription of the mRNA 2. It is assumed that the overwhelming majority of the genes and ORFs 1 of an organism, i.e. approx. 85%, is expressed similarly under various conditions. If therefore the aim was to determine several expression patterns of the complete genome of an organism, two different expression patterns would each have only a portion of genes, which would be present in different concentrations.

The high number of different probes on a biochip permits direct comparison with one another of the gene expression patterns found in the different channels. Differences in the respectively measured intensity between the two channels may be due to:

a) different markers,

b) fluctuations in the concentrations of overall mRNA populations A and B from two experiments A and B, which are not due to differences in expression,

c) differing expressions of an ORF 1 under different conditions.

Since the overwhelming majority of ORFs 1 of Escherichia Coli K 12 are expressed equally under various conditions, the differences in intensity which may occur through points a) and b) of the list given above, may be balanced out with the aid of a normalisation of the determined intensity values. After this balancing, the differences between two gene expression pattern become clear. If the same experiment were to be conducted on a biochip with few probes, then for statistical reasons such balancing would on the other hand be hardly possible. It is therefore expedient to provide at least 1000 and preferably more than 3000 probes on a biochip for each different gene.

Such normalisation may for example be undertaken as follows:

in each case the sum of all intensity values determined in a measuring channel is formed,

the greater of the sums determined is divided by the smaller of the sums determined, to give a factor, and

the determined intensity values from which the smaller sum was formed are multiplied by the factor.

The evaluation is effected by forming the differences between the determined intensity values of the altogether weaker-intensity channel multiplied by the factor, and the determined intensity values of the other channel, and specifically for each individual probe spot.

The differences between the normalised, determined intensity values allow a qualitative statement to be made regarding the different expression of a gene under differing conditions. The determined intensity values also allow a statement on how much more a specific gene was expressed under condition set A than under condition set B. A biochip according to the invention may thus be used to give a quantitative statement by means of scaling.

Within the scope of the invention it is also possible to produce a gene expression pattern of Escherichia Coli K 12 in which only one Escherichia Coli K 12 culture is analysed. The intensities measured in the probe spots of a biochip according to the invention may then referred to one another in qualitative terms and/or compared with expression patterns in a library.

It lies within the scope of the invention to employ probes with probe-lenghts of between 30 and 40, 40 and 50, 50 and 60, 60 and 70 or 70 and 80 bases.

Within the scope of the invention it is also possible to apply to one chip, probes optimised for Escherichia Coli K 12 with probes optimised for other Escherichia Coli K 12 strains or Escherichia Coli K 12 mutants, and/or to optimise together the probes provided for this purpose using the method described above.

1. Biochip with a multiplicity of probe spots wherein a probe is applied in each probe spot, each probe has a multiplicity of probe molecules, wherein
the probe sequences are nucleotide sequences,
the probe sequences have a base length of 30-80 bases,
the nucleotide sequences are made of ex-situ synthesised oligonucleotide sequences, and
at least one of the probes has a probe sequence which is identical or complementary to a section of an Open Reading Frame of Escherichia Coli K 12.

2. Biochip according to claim 1 wherein the majority of the probes has probe sequences which are identical or complementary to a section of an Open Reading Frame of Escherichia Coli K 12.

3. Biochip according to claim 2 wherein several probe sequences have a zone which is in each case identical or complementary to a section of the sequence zones SB 1-SB 12812:

4. Biochip according to claim 2 wherein several probe sequences have a zone which is in each case identical or complementary to one of the core zones KB 1-KB 12812.

5. Biochip according to claim 3 wherein several probe sequences have a zone which is in each case identical or complementary to one of the core zones KB 1-KB 12812.

6. Biochip according to claim 2 wherein said probe sequences being identical or complementary to the section of an Open Reading Frame of Escherichia Coli K 12 comprise a section which is identical or complementary to one of core zones KB 1-KB 12812.

7. Biochip according to claim 5 wherein said probe sequences being identical or complementary to the section of an Open Reading Frame of Escherichia Coli K 12 comprise a section which is identical or complementary to one of core zones KB 1-KB 12812.

8. Biochip according to claim 2 wherein the probe sequences have a base length of between 40 and 60 bases.

9. Biochip according to claim 7 wherein the probe sequences have a base length of between 40 and 60 bases.

10. Biochip according to claim 1 wherein the biochip has probes for more than ten different Open Reading Frames of Escherichia Coli K 12, with probe sequences in each case identical or complementary to a section of an Open Reading Frame.

11. Biochip according to claim 1,
wherein the oligonucleotide chip has probes for more than 100 different Open Reading Frames of Escherichia Coli K 12, with probe sequences in each case identical or complementary to a section of an Open Reading Frame.

12. Biochip according to claim 1,
wherein the biochip has probes for more than 1000 different Open Reading Frames of Escherichia Coli K 12, with probe sequences in each case identical or complementary to a section of an Open Reading Frame.

13. Biochip according to claim 1,
wherein the biochip has probes for more than 1000-4000 different Open Reading Frames of Escherichia Coli K 12, with probe sequences in each case identical or complementary to a section of an Open Reading Frame.

14. Biochip according to claim 1,
wherein the biochip has probes for more than 4000 different Open Reading Frames of Escherichia Coli K 12, with probe sequences in each case identical or complementary to a section of an Open Reading Frame.

15. Process for determining oligonucleotide sequences for a biochip, wherein sequences of a length of n bases from within an Open Reading Frame (ORF) are chosen and wherein it is tested whether the sequences exist only once within the corresponding ORF.

16. Process as claimed in claim 15, including one or more of the following steps:
testing whether the sequence is identical, partly identical to a predetermined extent or overlapping to a predetermined extent with other sequences or
testing whether the sequence has a GC-content within a predetermined range or
testing whether a hybrid formed by the sequence with it's complement has a calculated or otherwise determined melting temperature within a predetermined range or
testing whether the sequence is likely to form stable inter- or intramolecular hybrids with itself.

17. Process as claimed in claim 16, wherein at least one of the tests relating to a range (sequence-identity, sequence-overlap, hybrid melting temperature or GC content) is reiterated, said range is narrowed within a reiteration, and wherein reiteration is carried out until a predetermined amount of sequences per ORF is determined.

18. Process as claimed in claim 15, wherein the ORF is an ORF from within the Genome of Escherichia Coli K 12.

19. Process as claimed in claim 17, wherein the ORF is an ORF from within the Genome of Escherichia Coli K 12.
